(19) United States
(12) Patent Application Publication
(10) Pub. No.: US 2012/0238732 A1
(43) Pub. Date: Sep. 20, 2012

(54) OIL EXTRACTION FROM MICROALGAE

(75) Inventor: Tong Wang, Ames, IA (US)

(73) Assignee: IOWA STATE UNIVERSITY RESEARCH FOUNDATION, INC., Ames, IA (US)

(21) Appl. No.: 13/418,560

(22) Filed: Mar. 13, 2012

Related U.S. Application Data
(60) Provisional application No. 61/452,973, filed on Mar. 15, 2011.

(51) Int. Cl. C07K 1/36; C07K 14/00

(52) U.S. Cl. 530/418; 530/424; 530/350

(57) ABSTRACT

A method of oil extraction and biomass recovery from microalgae is disclosed. Methods according to the invention extract lipids from a biomass source and concentrate protein in the solid biomass source by alcohol processing. Aqueous alcohol processing methods provide extraction and separation techniques for lipids and protein-rich biomass suitable for biofuels.

### CROSS-REFERENCE TO RELATED APPLICATIONS

This application is a nonprovisional application of U.S. Provisional Application No. 61/452,973, filed Mar. 15, 2011, which is herein incorporated by reference in its entirety.

### FIELD OF THE INVENTION

The invention relates to lipid and biomass recovery from microalgae by aqueous alcohol processing methods. In particular, the extraction of lipids and precipitation of protein components of biomass are obtained from oleaginous microalgae. The methods according to the invention provide extraction and separation techniques for lipids and protein-rich biomass suitable for biofuels.

### BACKGROUND OF THE INVENTION

Currently, the species of microalgae selected for oil and biofuel production in the industry include various strains of Chlorella, Dunaliella, Nannochloropsis, Scenedesmus, and Schizochytrium. However, even within the same species, the oil (e.g. lipid) content varies significantly due to multiple factors, such as culture conditions and environment, and diversity in strains. The processing and fractionation characteristics of most, including these microalgae, have not been reported. These represent a few of the challenges for obtaining total lipid recovery from algae.

For the oil extraction from oilseeds, for example, effective cell breakage is the first step in oil recovery from microalgae. The cell wall of most microalgae is composed of cellulose, hemicellulose, and phospholipids. In Nannochloropsis and Chlorella, glucose is the major sugar after hydrolysis of polysaccharide of cell wall. Most microalgae, with the only exception of Dunaliella, have a rigid cell wall, which requires intensive energy to break and release cellular components. Studies also showed that for certain microalgae species, algaenan, a type of insoluble and non-hydrolysable biopolymer, constitutes the outer cell wall along with other polysaccharides. It is less than 1% in some Chlorella and is above 30% in certain Botryococcus strains. The chemical structure of algaenan has not been fully characterized; it is believed to include polyester, polyalcohol, polyaldehyde, or polyacid. The occurrence of such components (and others yet to be identified) presents a challenge for cell breakage.

Effective mechanical and chemical pretreatments are therefore needed to break the cells for oil extraction from microalgae. For standard oilseed processing and cell breakage, flaking or extrusion is used to break the cellular structure the dry seeds. Instead of extracting oil from dried and flaked or extruded algae cells with organic solvents as many research and development groups are exploring, we proposed to extract oil in algae's natural aqueous environment, then a further separation and purification of protein and cell wall components can be achieved in such system. Even though cell breakage is the essential first step in oil extraction, additional focus on oil distribution and extraction from broken cells is required.

Traditional lipid extraction procedure includes Soxhlet extraction (See AOAC Official Method 920.39), the Folch method (See Folch et al., G.H.S. (1957) A Simple method for the isolation and purification of total lipids from animal tissues. J. Biol. Chem. 226:497-509) and its modification such as Bligh and Dyer method (Bligh et al., (1959) A rapid method of total lipid extraction and purification. Canadian Journal of Biochemistry and Physiology, 37:(8) 911-917, 10.1139/o59-099). These references are incorporated herein by reference in their entirety. Still others have used extraction methods for algal lipids which include alcohols. For example, the use of 1-butanol, ethanol, and hexane/2-propanol mixtures for extraction of microalgal lipids have been employed from intact cells. However, the efficacy of such methods requires further improvement to obtain increased lipid extraction yields.

These organic solvent methods are analytical in nature and are not suitable for industrial algae oil extraction. Although hexanes is used for oilseed processing, the requirement for dry material and the fact of microalgae containing high level of polar lipids make it unsuitable and ineffective for algae oil extraction. Other physical methods, such as extrusion-expelling and supercritical CO₂ extraction that also requires dry mass are not effective or economically feasible because of the energy-intensive mechanical drying or the inefficient and possibly degradative sun drying. New techniques of oil extraction from microalgae's natural aqueous system include the use of acoustics, sonication, electromagnetic pulses, electromechanical lysing, nanoparticles, amphiphilic and switchable solvents. Even though many of these novel techniques have been reported in the media, information on their mechanisms and oil recovery yields are limited. Schizochytrium algae used for DHA production can be processed by drying and solvent extraction. DHA's high value and the low polar lipid content in this microalga justify for such extraction. For biofuel production, cost-effective and energy-efficient lipid extraction technologies still need to be developed.

Accordingly, it is an objective of the claimed invention to utilize quantification of the distribution of lipid and non-lipid biomass during an aqueous enzyme-assisted processing (AEP) of Nannocholoropsis and Schizochytrium.

A further object of the invention is to develop methods to efficiently fractionate lipid and non-lipid biomass by an ethanol and sonication-assisted processing (ESP).

A further object is to develop effective methods for fractionation by using alcohol and an alcohol process to improve upon the pure aqueous means of oil and biomass fractionation for algal oil recovery.

A further object is to utilize various species of microalgae in processing for commercial use in oil and biofuel applications.

### BRIEF SUMMARY OF THE INVENTION

The present invention provides efficient separation methods to extract lipid and precipitate protein from cell biomass, including oleaginous microalgae. In particular aqueous alcohol processing methods are used for the co-precipitation of the protein components of biomass sources that are initially disrupted by sonication in order to disrupt cellular structure of the microalgae. Alcohol unexpectedly performs the dual function of extracting lipid and precipitating protein components according to the invention. As a result, protein can be further extracted from the defatted cell biomass.

According to the invention, the subsequent use of concentrated alcohol at elevated temperatures permits more than merely lipid extraction from the emulsions. Beneficially, as a result of the disrupted cellular structure of the microalgae, the alcohol treatment permits further fractionation of the remaining components of the biomass source, including both carbohydrate and protein fractionation. Embodiments of the invention include both one-step and two-step separation methods for extracting lipid and co-precipitating protein from biomass sources.

Accordingly, an advantageous method of the invention provides for co-fractionating lipid and protein from a biomass source which may comprise, consist of and/or consist essentially of introducing an oleaginous microalgae biomass source in aqueous environment, lysing cell structure of said biomass source using sonication to generate an algal suspension, introducing an ethanol (ethyl alcohol) solvent at an elevated temperature, preferably close to the boiling point of the system to de-stabilize the algal suspension, separating a solid phase and an ethanol phase by centrifugation for further recovering protein and lipid in the two fractions.

In a further embodiment of the present invention a method for co-fractionating lipid and protein from a biomass source may comprise, consist of and/or consist essentially of introducing an oleaginous microalgae biomass source, lysing cell structure of said biomass source using sonication to generate an algal suspension, dewatering said algal suspension using a 50% ethanol (ethyl alcohol) solvent to recover a liquid and a solid phase, introducing an ethanol (ethyl alcohol) solvent at an elevated temperature to the solid phase for further fractionization of the biomass source, separating a solid phase and an ethanol phase by centrifugation for further recovery of protein and lipid components.

According to an embodiment of the invention an ethanol (ethyl alcohol) solvent is added to algal suspensions to reach a final concentration of 95% ethanol. Further, centrifugation is used to separate a solid phase and an ethanol phase for further recovery of protein and residual lipid content. The lipid and protein components can be further separated and purified.

Beneficially, the methods of the present invention do not require the use of enzymes for cell lysis in combination with the sonication. In addition, the costly and energy intensive step of drying, flaking and/or extruding as pretreatment steps for lipid removal is obviated. The methods of the present invention do not require the use of a supercritical fluid.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an embodiment of the invention depicting a procedure of enzyme and sonication-assisted processing (AEP) for lipid recovery from two microalgae. Nanno, Nannochloropsis; Schizo, Schizochytrium. Enzyme 1 is a mixture of cellulase/hemicellulose and lysophospholipase, and Enzyme 2 is a mixture of protease and phospholipase A2. Dotted line indicates alternative treatment.

FIG. 2 shows an embodiment of the invention depicting a one-step hot ethanol sonication-assisted processing (ESP) for lipid recovery from two microalgae. Nanno, Nannochloropsis; Schizo, Schizochytrium.

FIG. 3 shows an embodiment of the invention depicting a two-step hot ethanol sonication-assisted processing (ESP) for lipid recovery from Schizochytrium.

FIGS. 4A-F show cell breakage under 100× optical microscope for the two microalgae species used. Nannochloropsis (FIG. 4A, intact cells; FIG. 4B, enzyme treated cells; FIG. 4C, sonication and enzyme treated cells). Schizochytrium (FIG. 4D, intact cells; FIG. 4E, enzyme treated cells; FIG. 4F, sonication and enzyme treated cells).

FIGS. 5A-B show embodiments of the invention depicting lipid and biomass recovery from two microalgae as affected by scale and type of ESP processing.

FIGS. 6A-B show embodiments of the invention showing lipid distribution of two microalgae as affected by one-step (FIG. 6A, 50 g paste) and two-step (FIG. 6B, 20 g paste) ESP processing. Nanno, Nannochloropsis; Schizo, Schizochytrium.

FIG. 7 shows an embodiment of the invention showing the effect of acid hydrolysis time on lipid recovery of intact and sonicated microalgae cells.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE PREFERRED EMBODIMENT

The methods of the invention improve significantly upon the extraction methods previously employing alcohols for the extraction of algal lipids. For example, the use of 1-butanol, ethanol, and hexane/2-propanol mixtures for extraction of microalgal lipids have been employed from intact cells. The efficacy of such methods failed to achieve near-complete extraction of algal lipids. The methods of the present invention further employ the fractionation of other non-lipid components of microalgae, requiring the initial cell breakage step of the invention that inevitably forms a stable emulsion. Unexpectedly, the use of alcohol serves two purposes: providing an effective agent for protein precipitation (breaking the emulsion); and providing complete lipid solubilization at elevated temperature.

Methods of Oil Extraction

In an aspect of the invention separation methods to co-extract lipid and precipitate protein from cell biomass, including oleaginous microalgae biomasses are provided. As used herein the term co-extract and co-fractionate shall be understood to refer to the beneficially combined separation and extraction of both the lipid and protein contents of a biomass source. The methods of the invention beneficially obtain lipid extraction from the cell masses and improved biomass precipitation and recovery in the solid or cake fraction of the biomass sources compared to the results by using a pure aqueous system. In an aspect of the invention at least about 60% total lipid recovery is achieved using the methods of the invention. In a more preferred aspect of the invention, at least about 65% total lipid recovery is achieved, preferably at least about 70%, more preferably at least about 75%, still more preferably at least about 80%, still more preferably at least about 85%, and still more preferably at least about 90%. In a most preferred aspect of the invention the methods of the invention obtain at least about 95% total lipid recovery. The amounts of lipid recovery referenced herein refer to both one and two-step methods according to the invention.

In an aspect of the invention the alcohol process of the invention provide methods of lipid recovery generating at least about twice the lipid recovery achieved by traditional aqueous means of oil and biomass fractionation, preferably at least about three times the lipid recovery, and still more preferably at least about four times the lipid recovery.

As referred to herein, cell biomass includes all non-lipid components in algae. Various algae species can be utilized according to the invention, including for example Nannochloropsis, Nannochloris, Stichococcus, Neochloris oleoabundans, Hantzschia, Schizochytrium, Chlorella, Dunaliella, Botryococcus braunii, and Scenedesmus. Combinations of algae species may be utilized according to the methods of the invention as well. The biomass sources, including algae species disclosed herein are a non-limiting list of sources suitable for use according to the invention.

As described herein the methods of the invention provide for co-fractionating lipid and protein from a biomass source which may comprise, consist of and/or consist essentially of introducing an oleaginous microalgae biomass source in aqueous environment, lysing cell structure of said biomass source (i.e. sonication) to generate an algal suspension, introducing an alcohol solvent at an elevated temperature, preferably close to the boiling point of the system to de-stabilize the algal suspension, and separating a solid phase and an ethanol phase by centrifugation for further recovering protein and lipid in the two fractions.

In a further embodiment of the present invention a method for co-fractionating lipid and protein from a biomass source may comprise, consist of and/or consist essentially of introducing an oleaginous microalgae biomass source, lysing cell structure of said biomass source using sonication to generate an algal suspension, dewatering said algal suspension using a 50% ethanol (ethyl alcohol) solvent to recover a liquid and a solid phase, introducing an ethanol (ethyl alcohol) solvent at an elevated temperature to the solid phase for further fractionization of the biomass source, separating a solid phase and an ethanol phase by centrifugation for further recovery of protein and lipid components.

An optional step according to the various methods of the invention may include an initial determination of oil distribution with the biomass cellular structure. This may include, for example, determining the lipid content through use of quantification methods described in the Examples of the invention. Beneficially, according to the invention the identified oil distribution provides for more efficient fractionation of the lipid and non-lipid biomass by the alcohol and sonication-assisted processing according to the invention. In a further aspect of the invention, the step of first determining oil distribution within a biomass source is not required.

Cellular Disruption

In an aspect, for biomass disruption the oleaginous microalgae biomass source is first introduced in an aqueous environment. The cellular disruption occurs in the biomasses' (e.g. algae's) natural aqueous environment in order to efficiently extract oil. According to further aspects of the invention, aqueous biomass sources are initially disrupted in order to disrupt cellular structure of the microalgae. Disruption according to the invention includes the lysing of the cell structure of the microalgae. In a further aspect, disruption is utilized to generate an algal suspension.

In a preferred aspect, biomass disruption is achieved by sonication. For example, sonication treatment is provided for about 20 grams of cell paste with solid content of about 10-15% for about 4-10 minutes using a sonicator. A suitable sonicator for use according to the invention is commercially-available from Misonix Inc, Newtown, Conn. Additional features of a sonicator for use according to the invention includes a sonicator equipped with a probe (½″ tip) that is set at amplitude of 10 with output energy of 300 W at 20 kHz. Alternatively, sonication with greater than 1000 J can be applied to 10 mL of cell suspension with cell concentration of 1-15 g/L to achieve full cell disruption. Preferably, prolonged sonication treatment results in high degree of cell breakage. One of ordinary skill in the art will ascertain various variables for use of the sonication step according to the invention. For example, the sonication energy input needed for various cell types, cell densities, and sample volumes is within the level of ordinary skill in the art and such variables are considered within the scope of the invention.

Alcohol Processing

One-Step Methods

In a particular aspect, aqueous alcohol processing methods are used for the co-precipitation of the protein components of biomass sources after the cellular disruption is achieved. As set forth according to the invention, alcohol unexpectedly performs the dual function of extracting lipid and precipitating protein components. As a result, protein can be further extracted from the defatted cell biomass. According to the invention, any alcohol is utilized for the co-precipitation. According to the invention, alcohol is used to de-emulsify and/or destabilize the algal suspension.

In a preferred aspect, ethanol alcohol (ethyl alcohol) is utilized for the co-precipitation of the protein components of biomass sources. As one skilled in the art will ascertain based upon the beneficial disclosure of the present invention, additional alcohols may be employed.

According to an embodiment of the invention an alcohol solvent is added to the algal suspension. In one aspect, concentrated alcohol solvents re used. Preferably, concentrated alcohol solvents are between about 70-95% concentration according to the invention. Preferably concentrated alcohol solvents are added to the algal suspension to obtain a final concentration of at least about 80-95% alcohol, preferably at least about 85%-95% alcohol, more preferably at least about 90%-95% alcohol, and still most preferably about 95% ethanol (ethyl alcohol).

According to a further aspect of the invention, the concentrated alcohol solvents are introduced at elevated temperatures. In an aspect, the concentrated alcohol solvents are introduced at temperatures of at least about 60° C., preferably at least about 70° C., and more preferably from about 70-80° C. Beneficially, the use of alcohol at elevated temperatures effectively separates algae oil from other cellular components. In an aspect of the invention, an alcohol solvent is provided at elevated temperatures that approach or reach the particular boiling point of the system to de-stabilize the algal suspension. According to the invention, use of elevated temperatures unexpectedly permit more than merely lipid extraction from the emulsions. Beneficially, as a result of the initially disrupted cellular structure of the microalgae, the alcohol treatment permits further fractionation of the remaining components of the biomass source, including both carbohydrate and protein fractionation.

Two-Step Methods

Embodiments of the invention include both one-step and two-step aqueous alcohol treatments for extracting lipid and co-precipitating protein from biomass sources. In an aspect of the invention, a first dewatering step for the algal suspension is employed in the two-step aqueous alcohol treatment. A dewatering step may be included when biomass sources containing high water contents are employed, as a result of the increased amount of alcohol that would be required to obtain the preferred, final alcohol concentration of 95%. To eliminate the need for excessive quantities of alcohol to achieve the preferred, final alcohol concentration of 95%, an initial dewatering step is included. The dewatering step provides improved production feasibility to minimize the amounts of alcohol (e.g. ethanol) required according to the invention.

In an exemplary embodiment, a dewatering step may include use of about 50% alcohol (e.g. ethyl alcohol) solvent to recover a liquid and a solid phase. The dewatering step may be provided at ambient temperatures for a period of time sufficient to dehydrate the paste, so less amount of pure alcohol will be used to achieve high ethanol concentration in the extraction step. In an aspect of the invention, the dewatering step proceeds for at least 10 minutes, preferably at least 30 minutes. Thereafter, the aqueous biomass source is separated into solid and aqueous phases (e.g. centrifugation for a sufficient period of time according to sample size).

In the event a two-step aqueous alcohol treatment method is employed, upon separation of the two phases, the subsequent step of introducing an alcohol, such as ethanol (ethyl alcohol) solvent, at an elevated temperature and concentration to the solid phase for further fractionization of the biomass source is employed. The subsequent step may include the use of a concentrated alcohol solvent added to the algal suspension to obtain a final concentration of 95% alcohol, preferably 95% ethanol (ethyl alcohol) for separating the solid phase and alcohol phase by centrifugation for further recovery of protein and lipid components.

Separation

Methods of separation according to the invention are well appreciated by those skilled in the art, including for example the separation of a solid phase and an aqueous phase by centrifugation. In an aspect, the separation provides a solid phase and an aqueous alcohol (e.g. ethanol) phase by centrifugation. In an aspect of the invention, centrifugation under temperatures of at least about 60° C.-90° C., preferably about 80° C. is provided. The time required for the centrifugation of a particular biomass source may vary. In certain embodiments, centrifugation under heated temperatures for at least about 5 minutes, preferably at least about 10 minutes. One of ordinary skill in the art will ascertain the variables for the separation step according to the invention, including centrifugation. For example, the centrifugation timing, temperature and rate will vary based upon various cell types, cell densities, and sample volumes, such factors are within the level of ordinary skill in the art and considered within the scope of the invention.

Upon centrifugation the solid phases can be further extracted, separated and/or purified, as one of skill in the art will appreciate and are embodied within the scope of the present invention. According to the invention, the lipid and protein components (e.g. solids) obtained after separation from the aqueous phase by centrifugation, can be further extracted, separated and/or purified.

Enzymes and Other Pretreatment Steps

Beneficially, the methods of the present invention do not require the use of enzymes for cell lysis in combination with the sonication. In addition, the costly and energy intensive step of drying, flaking and/or extruding as pretreatment steps for lipid removal is obviated according to the methods of the invention. The methods of the present invention do not require the use of a supercritical fluid.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

An objective of experimentation according to the invention was to fully extract lipids from oleaginous microalgae. In particular, a further objective was to determine lipid and biomass distribution during aqueous and alcohol processing of oleaginous microalgae, with the intention of fully extracting lipid from the dewatered cell paste. Two species, Nannochloropsis and Schizochytrium were used in our aqueous and alcohol processing systems. The distribution of lipids and non-lipid components, i.e. biomass, during aqueous process and ethyl alcohol process were investigated. It was shown that majority of the lipids, 73% in Nannochloropsis and 87% in Schizochytrium were held by the broken cell mass in the centrifugation solids by aqueous processing. This retention of the lipid by the cell solids obtained by centrifugation demonstrates the need for developing a more effective lipid extraction method.

As shown in the results below, the use of an alcohol—ethanol—under elevated temperature was analyzed and allowed efficient extraction of lipid from the cell mass and at the same time improved biomass precipitation and recovery in the solid or cake fraction. This demonstrates that the separation of lipids from the cell mass. In addition, at the mean time improved biomass precipitation or recovery in the solid fraction was achieved. More than 68 and 95% total lipids were recovered on 50 g-scale (as-is paste basis) from Nannochloropsis and Schizochytrium. The at least 65% biomass recovery in the solid fraction for both algae from the alcohol process of the invention is superior to the control biomass recovery, 38 and 16% for Nannochloropsis and Schizochytrium by the aqueous process. As shown herein, pure aqueous means of oil and biomass fractionation is far less effective than fractionation by using alcohol, and alcohol process shows a great promise in algal oil recovery.

Microalgae. Nannochloropsis sp and Schizochytrium limacinum fresh cell pastes with 12% and 10% solid content were used for this study. The Nannochloropsis was obtained from Seambiotic Ltd. (Tel Aviv, Israel) and is a commercially-available strain, and the Schizochytrium (ATCC #MYA 1381) was grown in a bioreactor with glycerol as carbon source by a faculty colleague at Iowa State University. The lipid content was 22.7 and 50.1% dry weight basis (dwb) for Nannochloropsis and Schizochytrium, respectively, as determined by the method described below. The four enzymes used in AEP were obtained from Genencor (Copenhagen, Denmark) and their optimal conditions are given in Table 1. All enzymes were used under their optimal conditions. Absolute ethanol used in ESP was purchased from Fisher Scientific (Pittsburgh, Pa.). All other reagents were obtained from Sigma-Aldrich (St Louis, Mo.) unless otherwise noted.

TABLE 1
Enzymes used in aqueous enzyme-assistant processing (AEP)

| | | | Optimal | Amount used, |
|---|---|---|---|---|
| | Commercial | Optimal | temperature, | relative to dry |
| Enzyme | name | pH | ° C. | mass, % |
| Cellulase/ | Optimase | 4.6-5.0 | 50-60 | 5 |
| hemicell | CX 15L | | | |
| Protease | Protease 6L | 8-9 | 40-50 | 5 |
| Phospholipase | Multifect | 8.5 | 40-50 | 5 |
| A2 | LI 10L | | | |
| Lyso- | G-zyme G999 | 4.5 | 50 | 5 |
| phospholipase | | | | |
| (LysoPLA1) | | | | |

Quantification of Total Lipids in Microalgae.

Fresh microalgae paste with dry matter about 1 g was oven dried at 105° C. for 2 hr. The dry mass was ground using a mortar and pestle with the aid of solid carbon dioxide. Chloroform (16 mL) and methanol (8 mL) were added to algae sample and mixed for 1 hr. The mixture was then transferred to a separatory funnel where 5 mL water was added. The sample was gently mixed and given enough time for phase separation with occasional mixing. The lower solvent phase was collected and another portion of 20 mL chloroform was added to the upper layer. The chloroform phase was then collected and combined with previous solvent phase. The solvent was rotary evaporated and the total lipid was dried in a vacuum oven. The resulting lipid was weighed and dissolved in chloroform:methanol (2:1, v/v). All lipid recovery data obtained from AEP and ESP procedures were calculated based on the total lipid content determined by this method.

Quantification of Total Solid Content in Microalgae.

Total solid content was determined by an oven drying method according to the AOAC official method 925.09.

Study of Lipid Distribution During AEP with Sonication Treatment.

The treatments for this experiment were two microalgae, enzyme treatment alone, and a combination of sonication and enzyme treatment. Lipid and non-lipid biomass in each fraction were quantified as described in details in the following sections.

AEP and Lipid Extraction and Quantification Procedure:

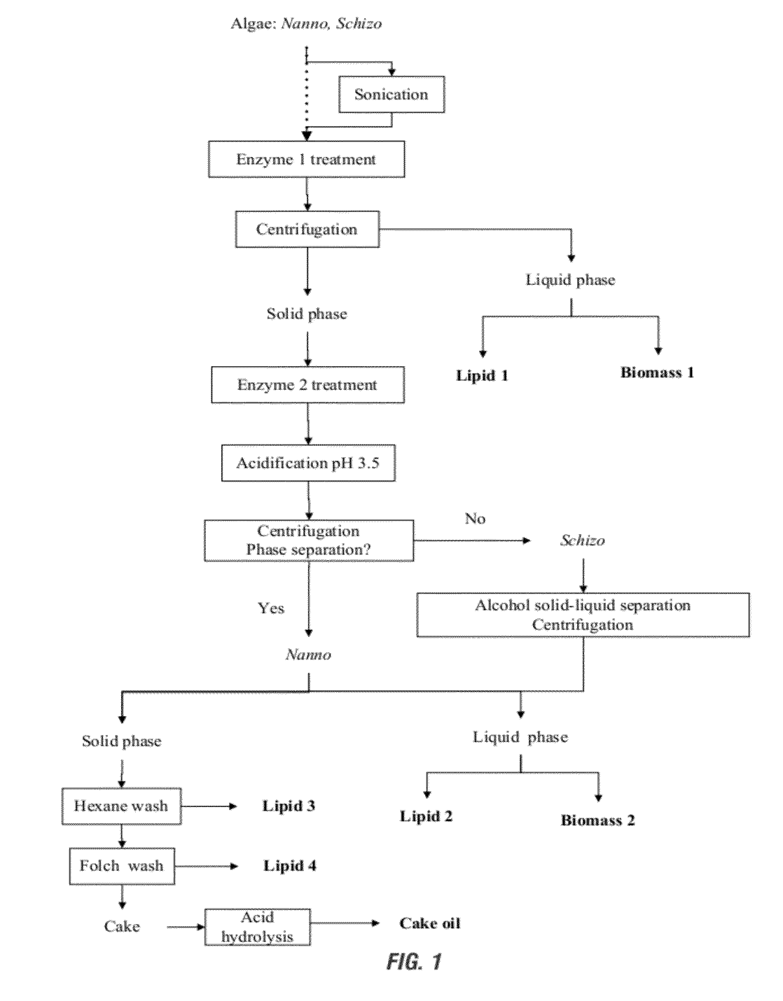

An outline of the AEP procedure is shown in FIG. 1. Each microalgae paste (20 g) was subjected to enzymatic hydrolysis. Optimase and lyso-phospholipase at 5% were added to each microalga (based on solid content of the system). Hydrolysis reaction was lasted for 24 hr at 50° C. at pH 4.5 with constant gentle shaking. After reaction, hydrolysates were centrifuged for 10 min at 6000×g. The supernatant phase was transferred to a separatory funnel for lipid extraction, and the quantified lipid was designated as Lipid 1 (quantification procedure details is described in the following section). The solid phase was dispersed into a second aqueous system at pH 8.5 with the same solid content as the initial sample (10-12%). Phospholipase A2 (5%) was added and the reaction lasted for 4 hr, followed by adding 5% protease 6 L. At the end of 24 hr, pH of the hydrolysates was adjusted to 3.5 to precipitate the proteins followed by another centrifugation at 6000×g for 10 min. Nannochloropsis had phase separation but Schizochytrium did not. Schizochytrium hydrolysate was then mixed with equal volume of absolute ethanol to cause solid precipitation. Such mixture was then centrifuged which led to clean phase separation. For both algae, the liquid phase was transferred to a separatory funnel for lipid extraction. The lipid in this liquid phase was extracted the same manner as for Lipid 1 and was designated as Lipid 2.

The solid phase was collected and dried for further lipid extraction and biomass quantification. The residual oil in the dried cake was extracted first by using 10-fold hexanes twice and the lipid was designated as Lipid 3. The cake was then further extracted by using 10-fold chloroform:methanol (2:1) twice and the lipid was designated as Lipid 4. Any residual lipid in the final cake after Lipid 3 and Lipid 4 removal was extracted by an acid hydrolysis method according to AOAC method 922.06, and the lipid obtained by acid hydrolysis was designated as Cake Oil.

Procedure of Lipid Extraction for Lipid 1 and Lipid 2 Quantification:

A mixture of chloroform and methanol was added to the liquid phase so that the ratio of chloroform: methanol: water in the funnel was 8:4:3 (v/v/v). After standing for phase separation, three layers appeared that were upper layer (water phase), intermediate layer (small emulsion phase), and the lower layer (organic phase). The lower layer was collected and saved. Ten-folds of chloroform was added to the reparatory funnel and mixed with the upper and intermediate layers. After another standing for phase separation, the amount of the emulsion phase decreased. Chloroform phase was collected and combined with the previous chloroform phase. The solvent was rotary evaporated and lipid was weighed and designated as Lipid 1 or Lipid 2. Emulsion phase and water phase were then separated. To quantify the lipid residue in emulsion, the emulsion phase was oven dried first and then lipid was extracted by 20-fold chloroform and ethanol (2:1, v/v) and its quantity determined.

Sonication Treatment:

Both Nannochloropsis and Schizochytrium (20 mL) with known solid content (12 and 10%, respectively) were placed in a 50 mL centrifuge tube. A laboratory ultrasonicator XL (Misonix Inc, Newtown, Conn.) equipped with a probe (½″ tip) was set at amplitude of 10 with output energy of 300 W at 20 kHz. The sample was treated for 5 min with ice-water bath surrounding the sample to avoid overheating. Samples were then subjected to enzymatic treatments as described above according to the experimental design.

Lipid Extraction with Ethyl Alcohol—the ESP Procedure.

The two microalgae were used to further explore the role of ethanol in lipid and biomass separation in aqueous system. Since prolonged sonication treatment resulted in high degree of cell breakage without the use of enzymes, enzymes were not used in this experiment. Ethyl alcohol was used in two different manners for Schizochytrium, and two different sample scales (5 g and 50 g cell paste, as-is basis) were used for oil extraction.

One-Step Process for Lipid Recovery and Biomass Fractionation:

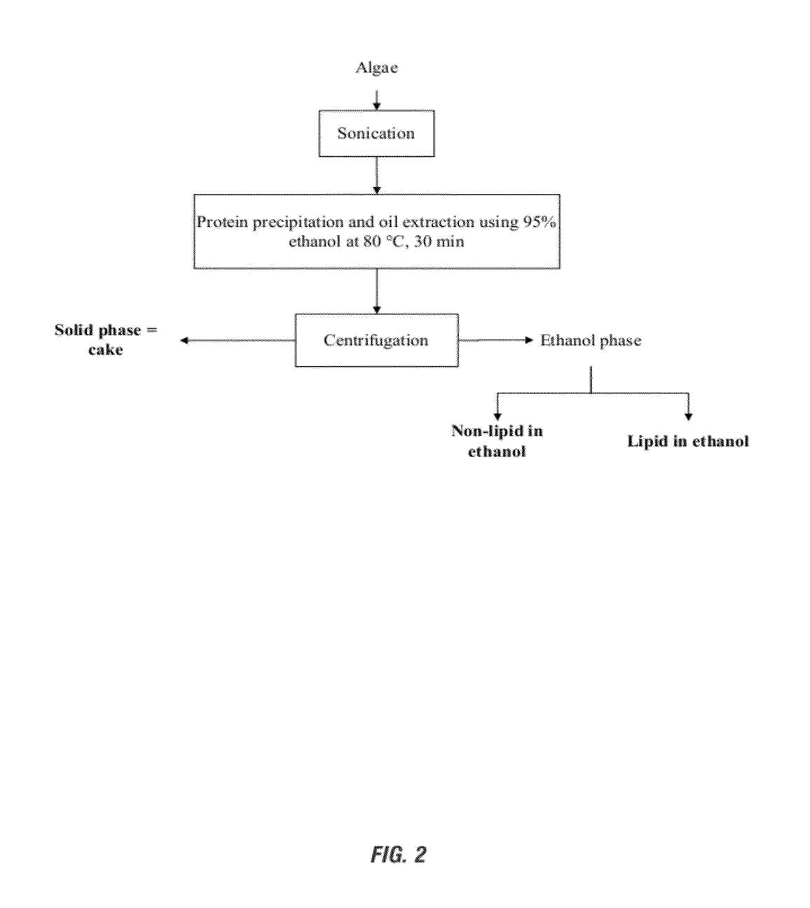

FIG. 2 outlines the process procedure for the one-step alcohol treatment. Nannochloropsis and Schizochytrium pastes were first subjected to ultrasonication treatment for 15 min as described previously in AEP processing. The resulting algal suspensions were mixed with a calculated amount of absolute ethanol under stirring to make the final ethanol concentration 95% (by volume). The mixture was then heated to 80° C. with reflux for 30 min, and was immediately followed by centrifugation at 3000×g for 10 min. The solid phase or precipitate was dried under the fumehood while the ethanol phase was de-solventized using a rotary evaporator. The non-lipid and lipid fractions in the ethanol phase were separated using a Folch's procedure as disclosed by Folch et al., J. Biol. Chem., 1957, 226:497-509 and Allard et al., Phytochemistry, 2000, 54:369-380, which are incorporated herein by reference in its entirety. Residual oil in both Cake and Non-lipid fraction of ethanol extract were quantified using acid hydrolysis method according to AOAC method 922.06.

Two-Step Process for Lipid Recovery and Biomass Fractionation of Schizochytrium:

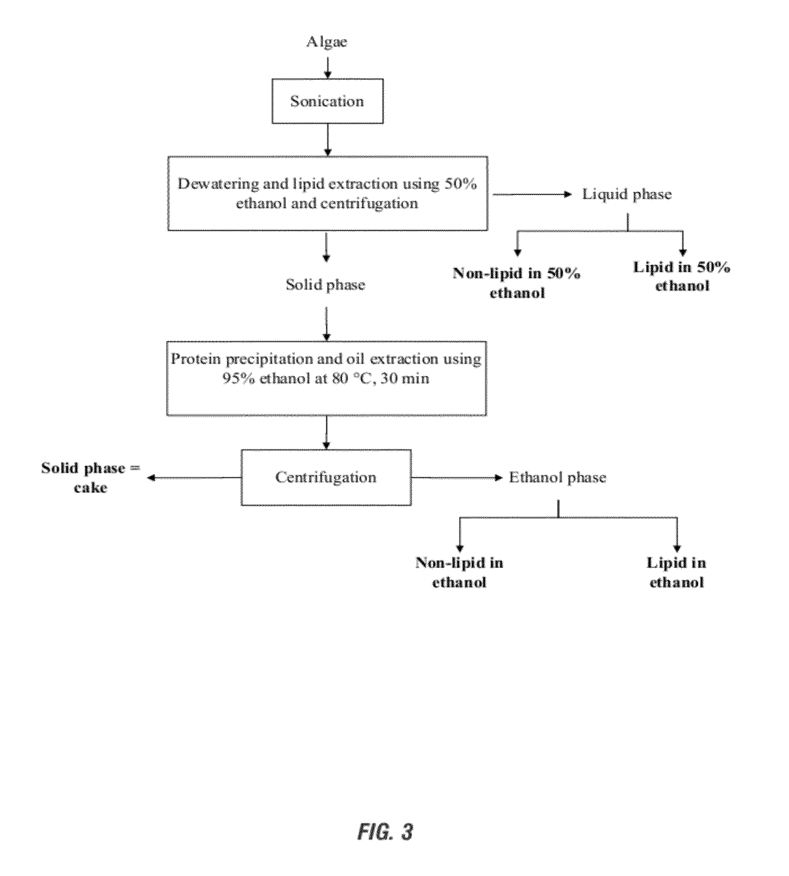

While all other steps were the same as one-step process, Schizochytrium (20 g call paste, as-is) after sonication treatment was first dewatered using 50% ethanol at ambient temperature (FIG. 3). The solid phase obtained by centrifugation at 1000×g for 5 min was used for subsequent 95% ethanol process and the ethanol lipid extract was dried and subjected to a Folch's procedure for non-lipid and lipid fractionation.

Calculation of Lipid and Biomass Recovery.

For each lipid fraction, the lipid recovery was calculated as the percentage of the weight of extracted lipids relative to total available lipids in the cells. The biomass recovery was calculated as the percentage of the dry weight of the non-extractable solids relative to the total cell mass with the extracted total lipids subtracted.

Protein Content Determination.

Protein contents of different algal fractions were quantified by the Dumas method using a rapid N III Nitrogen Analyzer (Elementar Americas, Inc. Mt. Laurel, N.J.), and was calculated as total nitrogen multiplied by a factor of 6.25.

Effect of Acid Hydrolysis on Lipid Recovery from Microalgae.

Both intact and sonicated cells of both algae species (original paste) were subjected to an acid hydrolysis before lipid extraction. All steps were the AOAC method 922.06 with varied hydrolysis time of 0, 15, 30, 60, and 120 min. This experiment was designed to test the effect of acid hydrolysis on lipid recovery or lipid degradation.

Statistical Analysis.

All experiments were conducted with duplicate treatments unless otherwise noted. Data analyses were done by using SAS program (version 9.1, SAS Institute Inc., Cary, N.C.). One-way Analysis of Variance (ANOVA) was used and Least Significant Differences were calculated at P of 0.05 (LSD_{0.05}).

### Example 2

Study of Lipid and Non-Lipid Biomass Distribution During AEP. Lipid Distribution During AEP:

As shown in FIG. 1, the lipid recovery from the aqueous phases during AEP processing is the sum of Lipid 1 and Lipid 2 fractions, which were the lipids partitioned in water. Lipid 3, 4, and cake oil fractions were the lipids that were trapped in the solids and had to be extracted by solvents with increasing polarity.

TABLE 2
Lipid (% of total) distribution through AEP processing

| | Nannochloropsis | | Schizochytrium | |
|---|---|---|---|---|
| Fraction\ | | Sonication/ | | Sonication/ |
| treatment | Enzyme | Enzyme | Enzyme | Enzyme |
| Lipid 1 | 17.5 ± 10.9 | 15.2 ± 13.2 | 6.7 ± 5.9 | 5.9 ± 1.7 |
| Lipid 2 | 8.7 ± 4.2 | 11.4 ± 4.8 | 5.9 ± 3.4 | 7.3 ± 6.3 |
| Lipid 3 | 3.4 ± 2.7 | 9.3 ± 2.8 | 19.8 ± 5.9 | 25.9 ± 0.3 |
| Lipid 4 | 19.5 ± 6.4 | 22.6 ± 2.1 | 9.1 ± 6.3 | 4.8 ± 1.2 |
| Cake Oil | 35.4 ± 6.2 | 24.6 ± 2.6 | 18.9 ± 10.0 | 36.9 ± 7.0 |
| Total Lipid | 84.4 ± 9.2 | 83.1 ± 10.5 | 60.4 ± 0.3^{b} | 80.9 ± 13.1^{a} |
| Recovery | | | | |

Means for each species within each row are not significant except for total recovery from Schizochytrium at P = 0.05.

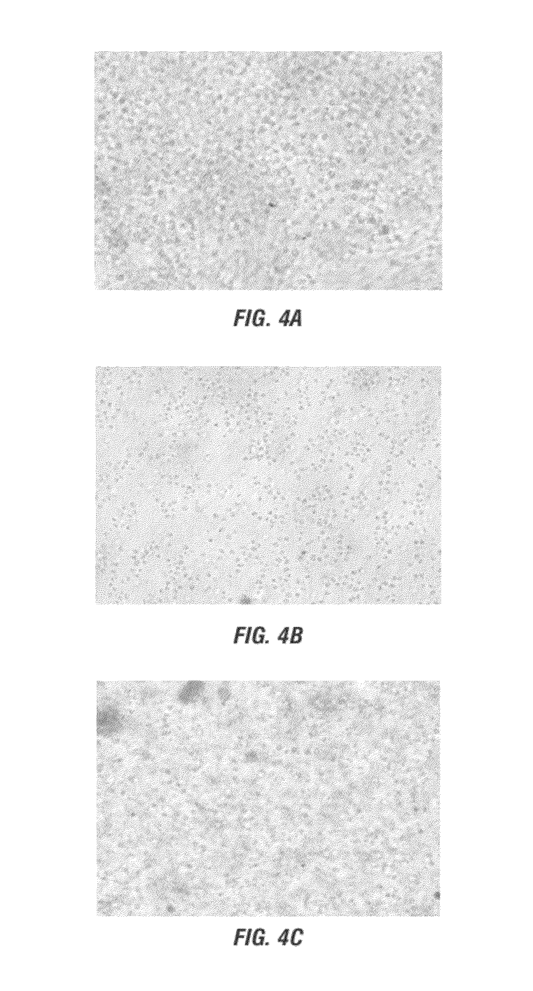

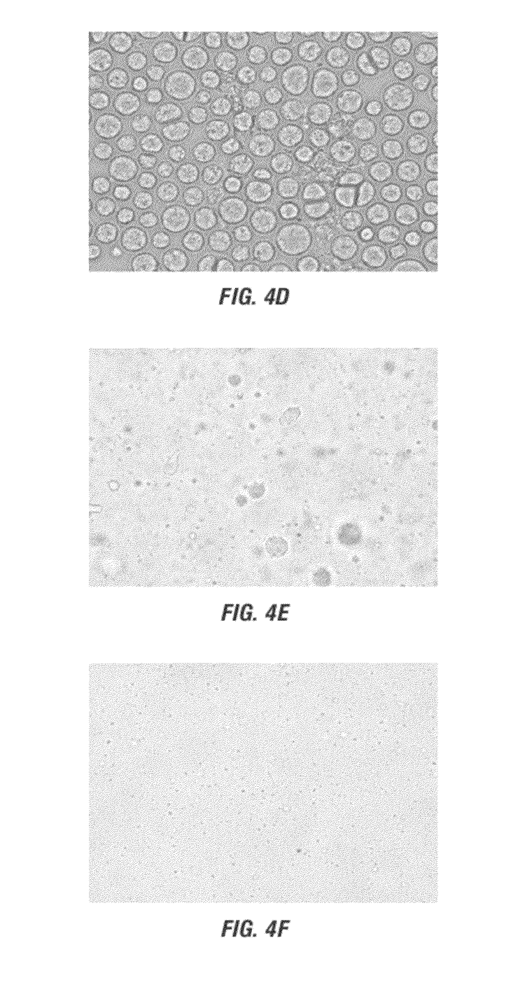

For Nannochloropsis, as shown in Table 2, no treatment effect (enzyme alone vs. sonication plus enzyme) was observed at the enzyme concentration and sonication conditions used. A microscopic observation (FIG. 4) indicates that a significant number of Nannochloropsis cells were still intact after both enzyme alone and sonication-enzyme treatments. Lipid recovery of AEP aqueous phases (sum of Lipid 1 and 2) was only 26.2% for enzyme alone treatment and 26.6% for sonication-enzyme treatment. The result suggests that more than 70% lipid was left in the cake which was held by other cellular components or in the intact cells. The subsequent hexane and chloroform:methanol extraction of the residue yielded 3.4 and 19.5% of total lipid for the enzyme treatment alone, and 9.3 and 22.6% of total lipid for sonication-enzyme treatment, respectively. Further lipid extraction from the cake by the acid hydrolysis procedure gave 35.4 and 24.6% of total lipid with the two treatments. These “cake” lipids were tightly bound to the solids or in the unbroken cells of the Nannochloropsis.

For Schizochytrium, as presented in the Table 2, the lipid recovery from aqueous fractions (Lipid 1 and 2) was only 12.6 and 13.2% for the enzyme and sonication-enzyme treatment samples, and sonication had no effect on this lipid recovery. Microscopic image (FIG. 4) showed that all cells were broken in sonication-enzyme treated samples while small amount of deformed cells were observed in the sample with enzyme treatment alone. This suggests that the combination of cellulose, hemicellulase, protease and phospholipases may be more effective in degrading the Schizochytrium cells than for those of Nannochloropsis. The rest of the lipid (more than 85%) was held in the cell debris under our experimental conditions. Different from Nannochloropsis where lipid in the cake can be much more easily extracted by chloroform:methanol rather than by hexanes, much more lipid (19.8% and 25.9%) in the cake of Schizochytrium could be extracted by hexanes than by chloroform:methanol (9.1% and 4.8%) for the two treatments. Further examination of the first water extractable lipids after cellulase and hemicellulase treatment also showed that more lipids can be extracted from Nannochloropsis than from Schizochytrium by water.

The different lipid extractabilities in the aqueous phases and with non-polar and polar solvents from the two microalgae may reflect the difference in lipid class composition between Nannochloropsis and Schizochytrium. Our lipid class quantification results of these two algae confirmed that Nannochloropsis contains more than 37% polar lipid (relative to total lipids) while Schizochytrium has much lower polar lipid content (14%). Polar lipids are relatively easily dispersible in aqueous phase. However, if they are held within the cell debris, they should be more easily extracted by the more polar solvents such as chloroform and methanol mixture than by hexanes. Often times the more exhaustive acid hydrolysis procedure has to be used to fully extract these polar lipids. In our future study, the lipid class composition of various lipid extracts will be fully characterized.

Nannochloropsis is an autotrophic microalga which uses solar energy to synthesize oil. Whereas, Schizochytrium is grown heterotrophically by converting the readily usable carbon (simple sugars and glycerol) to the storage lipids. Photosynthetic cells tend to contain more membrane structure therefore more polar lipids, as shown in this study. Thus, for oil extraction and the development of commercially feasible extraction methods, the type of algae and their polar and neutral lipid profiles should be fully studied and considered.

Although the enzyme treatments showed some promise, much more detailed studies are needed on this topic. Further enzyme screening and optimization either as singular treatment or in combination for each microalga should be conducted according to new knowledge and more understanding of the cell wall structure and chemical composition. For our research, since sonication treatment alone was effective to disrupt cells, we did not further investigate effectiveness and efficiency of enzyme treatment.

The lipids in the emulsion layers that were formed during Lipid 1 and Lipid 2 extraction totaled about 1.4 and 1.0% of total lipids in Nannochloropsis and Schizochytrium, respectively. These minor quantities were not included in the total lipid recoveries in Table 2. The large variations of the lipid distribution data may be due to the small quantity of algae cells used and the multiple-step treatments and processing of the samples. Nonetheless, general distribution trends were clearly observed.

The lipid distribution and lipid recovery results by AEP indicate that during lipid fractionation by AEP, strong interactions between lipid and other algal components, such as protein and carbohydrates, are formed and the oil is trapped or held, leading to low lipid recovery. An effective method with high lipid extractability is needed to increase lipid recovery.

Biomass Distribution of Two Microalgae During AEP Processing:

As shown in Table 3, the distribution of biomass or non-lipid components of Nannocholopsis is relatively even through AEP processing regardless of treatments. In Schizochytrium, about half of biomass (48.7-56.2%) was recovered from Lipid 1 aqueous phase, about one-third of total mass was in Lipid 2 aqueous fraction, and less than 15.7% was found in the final cake. This indicates Schizochytrium might have released more water soluble cellular components upon sonication and enzyme treatment. This biomass distribution pattern may not be desirable. Ideally, all non-lipid components should be recovered as solids then further separated.

TABLE 3
Biomass or non-lipid fraction distribution (% of total)
through AEP processing

| | Nannochloropsis | | Schizochytrium | |
|---|---|---|---|---|
| Fraction\ | | Sonication/ | | Sonication/ |
| treatment | Enzyme | Enzyme | Enzyme | Enzyme |
| After Lipid 1 | 27.6 ± 2.9^{b} | 37.2 ± 0.2^{a} | 56.2 ± 4.1^{a} | 48.6 ± 0.8^{b} |
| After Lipid 2 | 26.5 ± 0.0 | 33.3 ± 5.2 | 30.9 ± 2.3 | 35.8 ± 3.0 |
| Final cake | 45.9 ± 2.9^{a} | 29.4 ± 5.0^{b} | 12.9 ± 1.8 | 15.7 ± 2.2 |

Means within each row for each species are different when they have different superscripts at P = 0.05.
AEP, aqueous enzyme-assisted process.
After Lipid 1 or 2 indicates the biomass partitioned to the aqueous phase and with lipids removed.

### Example 3

Lipid Extraction with Ethyl Alcohol in Aqueous System. Lipid Recovery from Nannochloropsis by One-Step ESP Processing:

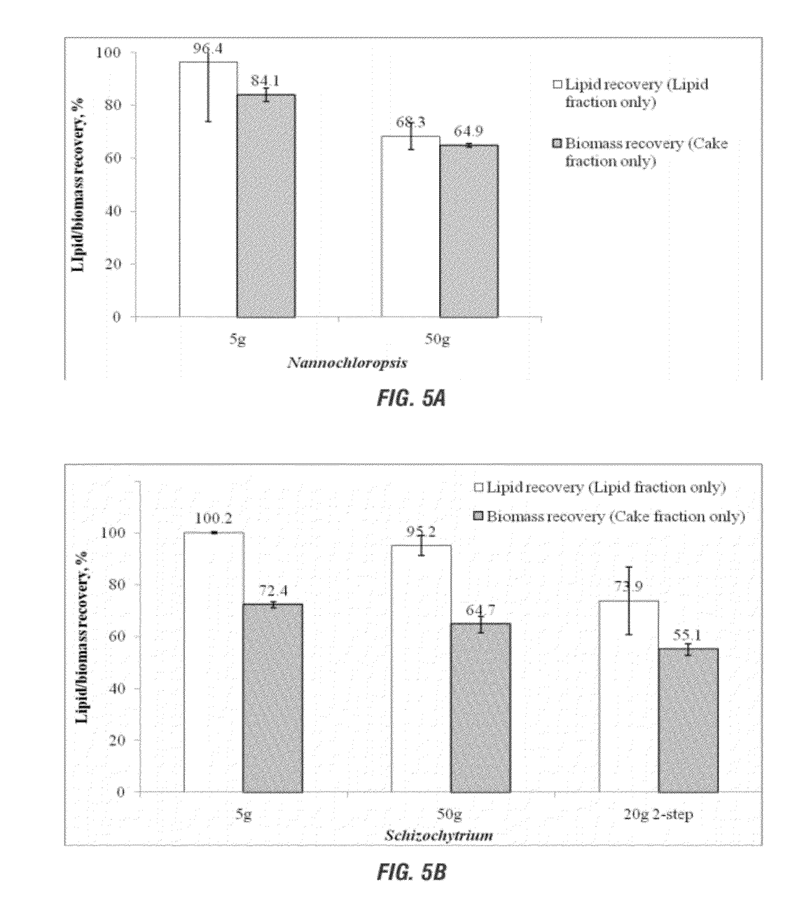

The lipid amount in Lipid fraction (FIG. 2) accounted for almost a full lipid recovery (96.4%, 5 g-scale in FIG. 5), which indicates hot ethanol treatment is a very effective extraction approach for sonicated algae samples. A scale-up procedure with 50 g of Nannochloropsis resulted in a lipid recovery of 68.3%, a value much lower than that of 5 g-scale. This is possibly due to the fact that larger amount of algal cells made sonication treatment difficult and insufficient to break all cells. Many sonication parameters can be optimized, and our current on-going research is focused on the study of sonication energy input needed for various cell types, cell density, and sample volume.

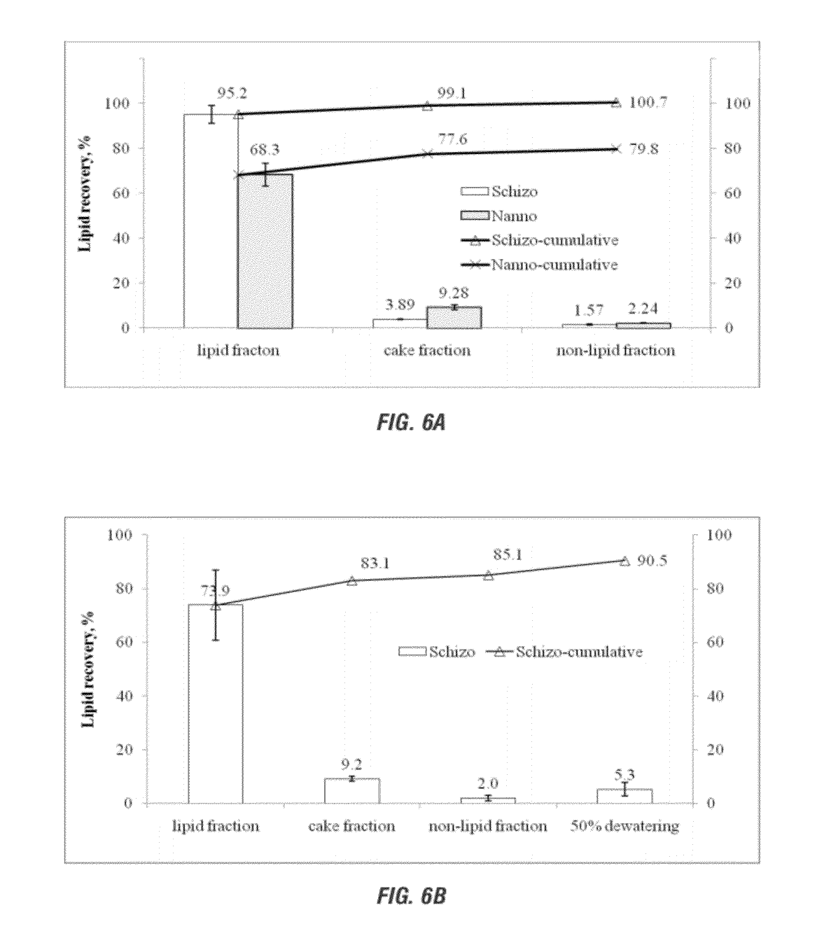

A further investigation examining the residual oil in both cake fraction and fractionated non-lipid fraction in ethanol extract revealed the lipid distribution of Nannochloropsis through one-step ESP, and the result is presented in FIG. 6. It is shown that scale-up ESP (50 g-scale) recovered only 79.8% total lipid, with the cake fraction held up 9.3% lipids, and the non-lipid fraction of the lipid extract accounted for 2.2% total lipids. There may be another reason for this low total lipid recovery from Nannochloropsis, as discussed in later section.

On the other hand, biomass recovery from Cake fraction is 84.1% and 64.9% for the 5 g-scale and 50 g-scale processing, and the rest of biomass was found in the ethanol phase (marked as Non-lipid fraction). The enrichment of biomass in Cake fraction instead of in aqueous phase as a result of ethanol treatment would make it desirable for subsequent fractionation of protein and carbohydrate.

Lipid Recovery from Schizochytrium by One-Step and Two-Step ESP Process:

The 5 g-scale treatment and 50 g-scale treatment for one-step ESP were studied similarly as for Nannochloropsis (FIG. 2). Because the starting microalgae samples contain 90% water and significant amount of ethanol is needed to make the mixture having 95% ethanol concentration. Therefore, production feasibility with such high quantity of ethanol requirement will be low. A two-step fractionation procedure was designed (FIG. 3) and it could be applied to both microalgae. However, it was observed that a clear liquid was able to separate from solid phase by centrifugation in Nannochloropsis after sonication, so the solid that contained the majority of lipids was effectively concentrated before alcohol extraction. Therefore, for this experiment only Schizochytrium was studied for two-step ESP.

The lipid content in Lipid fraction represents a total lipid recovery of 100.2% and 95.2% for 5 g-scale and 50 g-scale treatment (FIG. 5), respectively, during one-step ESP. Two-step ESP (20 g-scale) gave a lipid recovery of 73.9% in the 95% ethanol extract, which was much lower than one-step treatment. However, two-step ESP reduced ethanol usage by two-thirds. A further lipid distribution study showed that in two-step ESP, 5.3% of total lipid was in the 50% dewatering phase and 9.2% was trapped in the cake fraction, and only 2.0% was in the non-lipid fraction of 95% ethanol lipid extract (FIG. 6), with total recoverable lipids of 90.5%. In the future, a second 95% ethanol extraction with a small volume of the solvent from the solids can be done to yield higher total lipid recovery.

### Example 4

Biomass Distribution with One-Step and Two-Step ESP Processing of the Schizochytrium:

The protein content of various biomass fractions is presented in Table 4. As shown in Table 5, Biomass distribution of Schizochytrium in one-step ESP is similar to that of Nannochloropsis, with 64.7% in cake fraction and 35.3% in non-lipid fraction. In two-step ESP processing, 43.3% biomass was first fractionated into 50% dewatering ethanol phase, and 55.1% biomass was left in the cake while only 5.8% was in the 95% ethanol lipid extract. Both the 50% dewatering phase and cake are good biomass source in two-step ESP processing. For various biomass fractions, the protein content is presented in Table 4.

TABLE 4
Protein content in each biomass fraction along one-step
(50 g-scale) and two-step (20 g-scale) ESP processing

| | | Cake fraction, | Non-lipid | 50% dewatering, |
|---|---|---|---|---|
| Process | Species | % | fraction, % | % |
| One-step | Schizo | 54.6 ± 1.6^{a} | 30.3 ± 0.5^{b} | — |
| | Nanno | 36.9 ± 0.1^{a} | 14.1 ± 3.2^{b} | — |
| Two-step | Schizo | 44.2 ± 0.4^{a} | N/A* | 30.2 ± 0.1^{b} |

*Not enough quantity to measure protein content.
Means with different superscript for each species (within each row) are significantly different at P = 0.05.
Nanno, Nannochloropsis;
Schizo, Schizochytrium.
ESP, ethanol and sonication-assisted process.

TABLE 5
Biomass distribution along one-step (50 g-scale) and
two-step (20 g-scale) ESP processing

| | | Cake | | 50% alcohol | Total |
|---|---|---|---|---|---|
| | | fraction, | Non-lipid | dewatering, | recovery, |
| Process | Species | % | fraction, % | % | % |
| One-step | Schizo | 64.7 ± 3.1^{a} | 35.3 ± 2.0^{b} | — | 100.0 ± 1.2 |
| | Nanno | 64.9 ± 0.7^{a} | 34.4 ± 1.2^{b} | — | 99.3 ± 0.6 |
| Two-step | Schizo | 55.1 ± 2.2^{a} | 5.8 ± 1.6^{c} | 43.4 ± 4.4^{b} | 104.2 ± 0.6 |

In one-step ESP, means with different superscript for each species (within each row) are significant difference at P = 0.05;
In two-step ESP, means with different superscript represent significant difference at P = 0.05.
Nanno, Nannochloropsis;
Schizo, Schizochytrium .
ESP, ethanol and sonication-assisted process.

Effect of Acid Hydrolysis on Lipid Recovery from Microalgae:

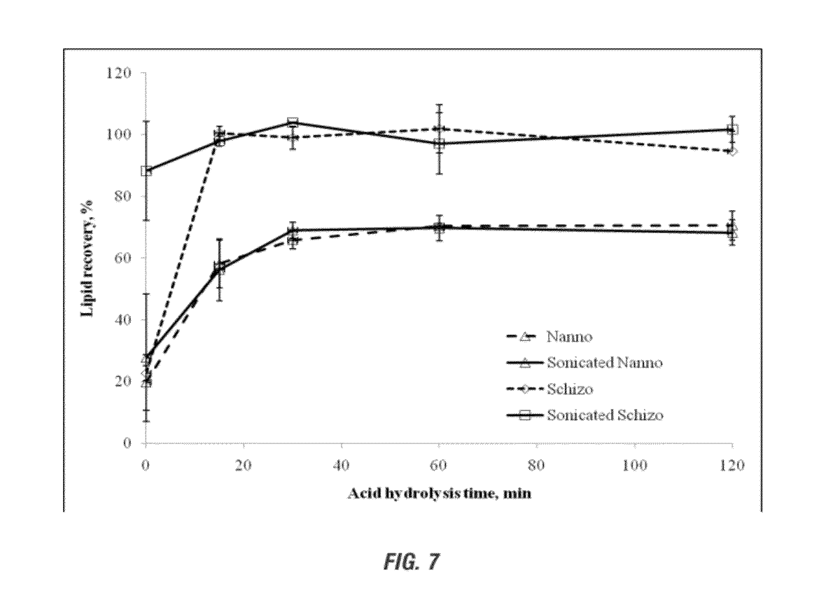

Since the total lipid recovery from the Nannochloropsis was not near 100%, even after the exhaustive acid hydrolysis treatment of the cell debris (cake), we designed a study to examine if longer hydrolysis time could result in higher lipid recovery. For this study, both microalgae were used with and without cell breakage by sonication treatment. As shown in FIG. 7, for different treatments and species, it was confirmed that the time of 30 min digestion as recommended by AOAC official method is sufficient to obtain the maximum lipid extraction and the extended heating did not improve lipid recovery. For Nannochloropsis, sonic lid not improve lipid recovery with direct acid hydrolysis extraction, shown as at zero time of acid incubation, the lipid recovery for both treatments was about 20%. For Schizochytrium, at zero time, sonication led to 90% lipid recovery, while only 20% lipid was extracted from the intact cells. Acid hydrolysis for up to 15 min of intact cells compensated the effect of sonication and at the end of 30 min hydrolysis, even intact cell treatment had near 100% lipid recovery. It indicates that a simple acid hydrolysis for Schizochytrium is sufficient to extract all the oil. Such acid hydrolysis procedure is suitable for analytical purpose, but not for commercial lipid extraction because of the high acid concentration used and the degradation of other cellular components.

The maximum lipid recovery from Nannochloropsis was only about 70% by the acid hydrolysis method. As explained previously, Nannochloropsis contains significant amount of polar lipids, especially glycolipids. The glycosidic bond is sensitive to acid hydrolysis conditions, so the hydrolyzed or degraded product from the various types of glycolipids, i.e. the diacylglycerols, is recovered in the lipid fraction. If this lipid fraction was compared to the total lipid that was extracted with non-destructive polar solvents, it will present a much lower lipid recovery, even if all the lipids are effectively recovered. This reasoning can also explain the much lower lipid recovery from the Nannochloropsis compared to that from Schizochytrium during AEP and ESP processing, when the residual lipids in the cell debris or the non-lipid fraction of the ethanol lipid extract were recovered by the acid hydrolysis procedure. For example, the lipid recovered by ethanol extraction from Nannochloropsis on 50 g-scale was 68.3% and the ultimate total lipid recovered from all fractions was 79.8% (FIGS. 5 and 6). This incomplete lipid recovery can be explained by acid hydrolysis of the polar lipids and thus an underestimation of the lipid recovery. A more detailed quantification of various amounts of polar lipids in various fractions will allow us to calculate theoretical recovery if acid hydrolysis has to be used in quantifying residual lipids in the non-lipid biomass products.

This is the first known systematic study and report that uses ethanol for microalgae lipid extraction and biomass co-precipitation, though ethanol has been used in corn oil extraction (Kwiatkowski et al., J. Am. Oil Chem. Soc. 2002, 79, 825-830). We have demonstrated that the approach of combination of sonication and hot ethanol treatment is an effective process for separating oil from other cellular components of microalgae without the energy intensive drying step. On the other hand, the lipids and biomass distribution during pure aqueous processing were proved to be undesirable and the pure aqueous method for lipid extraction was not effective compared to the alcohol extraction.

The inventions being thus described, it will be obvious that the same may be varied in many ways. Such variations are not to be regarded as a departure from the spirit and scope of the inventions and all such modifications are intended to be included within the scope of the following claims.

What is claimed is:

1. A method for co-fractionating lipid and protein from a biomass source comprising:
introducing an oleaginous microalgae biomass source in an aqueous environment;
lysing cell structure of said biomass source using sonication to generate an algal suspension;
introducing an ethanol solvent at an elevated temperature to de-emulsify or destabilize said algal suspension; and
separating a solid phase and an ethanol phase by centrifugation for further extraction of protein and lipid content; and
extracting lipid and protein components, wherein said method obtains improved lipid and protein fractionation compared to a pure aqueous method.

2. The method of claim 1 wherein said ethanol solvent is a concentrated ethyl alcohol added to said algal suspension to obtain a final algal suspension concentration of about 95% ethanol.

3. The method of claim 1 wherein said temperature of said ethanol solvent is between about 60° C. and about 80° C.

4. The method of claim 1 wherein said temperature of said ethanol solvent is approaching the boiling point of said solvent.

5. The method of claim 1 further comprising centrifugation after said de-emulsification to separate a solid phase and an ethanol phase for further extraction of protein and lipid content.

6. The method of claim 1 further comprising separation of said lipid and protein components.

7. The method of claim 6 further comprising the precipitation of protein from said lipid and protein components.

8. The method of claim 1 further comprising purification of said lipid and protein components.

9. The method of claim 1 wherein said sonication does not require use of enzymes for cell lysis.

10. The method of claim 1 wherein said biomass source is not dried, flaked or extruded.

11. The method of claim 1 wherein said heating step does not require use of a supercritical fluid.

12. The method of claim 1 wherein said oleaginous microalgae biomass source is a member selected from the group consisting of Nannochloropsis, Nannochloris, Stichococcus, Neochloris oleoabundans, Hantzschia, Schizochytrium, Chlorella, Dunaliella, Botryococcus braunii, Scenedesmus and combinations thereof.

13. A method for co-fractionating lipid and protein from a biomass source comprising:
introducing an oleaginous microalgae biomass source;
lysing cell structure of said biomass source using sonication to generate an algal suspension;
dewatering said algal suspension using a 50% ethanol solvent to recover a liquid and a solid phase;
introducing a concentrated ethanol solvent at an elevated temperature to said solid phase for further fractionization of said biomass source; and
separating a solid phase and an ethanol phase by centrifugation for further extraction of protein and lipid content wherein said method obtains improved lipid and protein fractionation compared to a pure aqueous method.

14. The method of claim 13 wherein said concentrated ethanol solvent is ethyl alcohol and is added at a temperature between about 60° C. and about 80° C. to said solid phase to obtain a final concentration of about 95% ethanol.

15. The method of claim 13 further comprising separation of said lipid and protein components.

16. The method of claim 13 further comprising purification of said lipid and protein components.

17. The method of claim 13 wherein said sonication does not require use of enzymes for cell lysis.

18. The method of claim 13 wherein said biomass source is not dried, flaked or extruded and/or wherein said heating step does not require use of a supercritical fluid.

19. The method of claim 13 wherein said oleaginous microalgae biomass source is a member selected from the group consisting of Nannochloropsis, Nannochloris, Stichococcus, Neochloris oleoabundans, Hantzschia, Schizochytrium, Chlorella, Dunaliella, Botryococcus braunii, Scenedesmus and combinations thereof.

20. The products produced according to the method of claim 1.

21. The products produced according to the method of claim 13.
